(19) United States
(12) Patent Application Publication
(10) Pub. No.: US 2022/0304847 A1
(43) Pub. Date: Sep. 29, 2022

(54) Reconfiguration Compatible Thermal Pad

(71) Applicant: C. R. Bard, Inc., Franklin Lakes, NJ (US)

(72) Inventors: Melody M. H. Kuroda, Durham, NC (US); Charles D. Shermer, Raleigh, NC (US); Christopher A. Basciano, Apex, NC (US)

(21) Appl. No.: 17/700,216

(22) Filed: Mar. 21, 2022

Related U.S. Application Data
(60) Provisional application No. 63/164,970, filed on Mar. 23, 2021.

(51) Int. Cl. A61F 7/00; A61F 7/08

(52) U.S. Cl. CPC A61F 7/00; A61F 7/08; A61F 7/007; A61G 2210/90; A61F 2007/0054

(57) ABSTRACT

Disclosed herein is a system, apparatus and method directed to an expandable and conformable targeted temperature management (TTM) pad. The system, apparatus and method pertain to a medical pad for exchanging thermal energy between a TTM fluid and a patient. The pad includes an insulating layer, a fluid containing layer for containing the TTM fluid, and a patient contact surface. The fluid containing layer is configured for circulating the TTM fluid. The patient contact surface defines a first patient contact area to facilitate thermal energy exchange with the patient. One or more slits are arranged in at least the insulating layer of the pad to facilitate stretching the pad and conforming to a contour of a patient's anatomy. The stretching can expand the patient contact surface to a larger second patient contact area. The pad can further include a flexible fabric cover.

### PRIORITY

This application claims the benefit of priority to U.S. Provisional Application No. 63/164,970, filed Mar. 23, 2021, which is incorporated by reference in its entirety in this application.

### SUMMARY

Briefly summarized, embodiments disclosed herein are directed to systems, methods, and apparatuses for an expandable and conformable medical pad for Targeted Temperature Management (TTM) procedures, that is, for cooling or heating a patient to provide medical benefits, such as neuroprotection following a stroke or surgery.

One problem that often arises with TTM systems is applying medical pads to accommodate patients of different sizes effectively and comfortably. An ill-fitting medical pad may hinder effective transmission of thermal energy between the pad and the patient, and may also give rise to patient discomfort. In an acute case, an improperly fitting pad could lead to overheating or overcooling the patient, and could potentially engender a risk of medical complications. As a result, conventionally, numerous sizes are available, but selecting the correct size may be difficult and complex, as well as requiring multiple sizes to be maintained in stock. Moreover, there exists a risk of selecting the wrong size pad and making multiple kit components unusable, resulting in waste of a valuable product and frustration on the part of users due to incorrect pad sizing. The disclosed embodiments of devices and methods can address this problem by adjusting the patient contact area of the thermal pad to better accommodate different patient sizes.

A second problem is that the TTM pads may be inflexible, and cannot conform to variations in patient anatomy. For example, the pad may not be flexible enough to curve with a short radius of curvature, and thus may not conform to contours of a particular patient's anatomy. This can result in poorer contact than would be indicated by the pad area, that is, less than the pad's full capacity for thermal exchange with the patient may be utilized. Some thermal energy may go to waste, even while the patient is heated or cooled inadequately. Having flexible components would permit better effective area contact for the available pad area and alleviate “bubbles” or gaps between the pad and the patient's body.

A third problem is irritation of patients' skin due to pressure from an edge of the medical pads of the TTM system. Specifically, the medical pads may have a harsh edge that may cause discomfort and irritation for some patients. Even though the pads can contain a pliable material, like a hydrogel, that can conform to the patient's skin and provide good thermal contact, some patients may experience skin irritation. In some cases, patients may use the pads for extended periods, exacerbating such discomfort and irritation after repeated contact. Embodiments of the disclosed apparatus and system can also address this problem.

Disclosed herein is a medical pad for exchanging thermal energy between a TTM fluid and a patient. The pad comprises an insulating layer, a fluid containing layer for containing the TTM fluid, and a patient contact surface. The fluid containing layer is configured for circulating the TTM fluid. The patient contact surface defines a first patient contact area to facilitate thermal energy exchange with the patient. One or more slits are arranged in at least the insulating layer of the pad to facilitate stretching the pad. The stretching expands the patient contact surface from the first patient contact area to a larger second patient contact area.

In some embodiments, one or more fluid containing layer slits are arranged in the fluid containing layer, the fluid containing layer slits coinciding with the one or more slits of the insulating layer.

In some embodiments, the one or more slits facilitate the patient contact surface conforming to a contour of a body part of the patient. The patient contact surface contacts the body part.

In some embodiments, the medical pad further comprises a flexible fabric cover. The flexible fabric cover further facilitates the stretching of the pad.

In some embodiments, the flexible fabric cover has a soft texture configured to reduce pressure on skin of the patient from an edge or surface of the pad.

In some embodiments, the flexible fabric cover comprises one or more of nylon, polychloroprene, elastane, latex, isoprene, polyisoprene, elastolefin, polybutadiene, nitrile rubber, butyl rubber, or a loose-woven fabric.

In some embodiments, the flexible fabric cover comprises an exterior border portion surrounding a perimeter of the pad, and a slit-covering portion coinciding with the one or more slits in the insulating layer.

In some embodiments, the flexible fabric cover facilitates the patient contact surface conforming to a contour of a body part of the patient, wherein the patient contact surface contacts the body part.

In some embodiments, the flexible fabric cover coincides with the one or more slits in the insulating layer, and an edge guard surrounds a perimeter of the pad, the edge guard configured to reduce pressure from an edge or surface of the pad on skin of the patient.

In some embodiments, the edge guard comprises one or more gaps coinciding with the one or more slits of the insulating layer.

In some embodiments, the edge guard has one or more edge guard slits coinciding with the one or more slits of the insulating layer.

In some embodiments, the edge guard includes a pliant edge guard cover, and a cushion filling contained within the pliant edge guard cover.

In some embodiments, the edge guard comprises silicone or another pliant shock-absorbent material.

In some embodiments, the fluid containing layer includes a plurality of tortuous fluid flow paths, and the one or more slits are arranged in portions of the insulating layer not coinciding with the fluid flow paths of the fluid containing layer.

Also disclosed herein is a method of providing a targeted temperature management (TTM) therapy to a patient. The method comprises providing a TTM system comprising a TTM module, a thermal pad, and a fluid delivery line (FDL). The TTM module is configured to provide a TTM fluid. The thermal pad is configured to receive the TTM fluid from the TTM module to facilitate thermal energy transfer between the TTM fluid and a patient. The FDL extends between the TTM module and the thermal pad. The FDL is configured to provide TTM fluid flow between the TTM module and the thermal pad. The thermal pad comprises an insulating layer and a patient contact surface defining a patient contact area to facilitate thermal energy exchange with the patient. One or more slits are arranged in at least the insulating layer of the pad. The method further comprises stretching the pad. The stretching is facilitated by the one or more slits in the insulating layer. The stretching expands the patient contact area from the first patient contact area to a larger second patient contact area. The method further comprises applying the pad to the patient. The method further comprises delivering TTM fluid from the TTM module to the thermal pad.

These and other features of the concepts provided herein will become more apparent to those of skill in the art in view of the accompanying drawings and following description, which disclose particular embodiments of such concepts in greater detail.

### BRIEF DESCRIPTION OF THE DRAWINGS

Embodiments of the disclosure are illustrated by way of example and not by way of limitation in the figures of the accompanying drawings, in which like references indicate similar elements and in which:

FIG. 1 illustrates a TTM system using medical pads for heating and/or cooling a patient, according to some embodiments;

FIG. 2 illustrates TTM medical pads being placed on a patient, according to some embodiments;

FIG. 3 illustrates a structure of an exemplary medical pad, according to some embodiments;

FIG. 4A shows a patient with an oversized TTM medical pad;

FIG. 4B shows a TTM medical pad inadequately conforming to a contour of a patient's anatomy;

FIG. 5A illustrates a TTM medical pad with slits and a flexible fabric cover, according to some embodiments;

FIG. 5B illustrates flexing of a TTM medical pad with slits and a flexible fabric cover, according to some embodiments;

FIG. 6 shows the TTM medical pad of FIG. 5B conforming to a contour of a patient's anatomy, according to some embodiments;

FIG. 7 illustrates a TTM medical pad with a flexible fabric cover surrounding its perimeter and covering slits, according to some embodiments;

FIG. 8A illustrates a TTM medical pad with an edge guard with gaps surrounding its perimeter, and a flexible fabric covering slits, according to some embodiments;

FIG. 8B illustrates a TTM medical pad with an edge guard with slits surrounding its perimeter, and a flexible fabric covering the slits, according to some embodiments;

FIG. 9 illustrates a TTM medical pad with a pattern of slits arranged away from its fluid containing layer, according to some embodiments;

FIG. 10A provides an exploded perspective view of a TTM fluid filter, according to some embodiments;

FIG. 10B provides a cross-sectional side view of the filter of FIG. 10A, according to some embodiments;

FIG. 10C provides a side cross-sectional view of the thermal contact pad of FIG. 3 incorporating the filter of FIG. 10A, according to some embodiments; and

FIG. 11 illustrates a method of using a TTM medical pad with slits and a flexible fabric cover, according to some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a probe disclosed herein includes a portion of the probe intended to be near a clinician when the probe is used on a patient. Likewise, a “proximal length” of, for example, the probe includes a length of the probe intended to be near the clinician when the probe is used on the patient. A “proximal end” of, for example, the probe includes an end of the probe intended to be near the clinician when the probe is used on the patient. The proximal portion, the proximal end portion, or the proximal length of the probe can include the proximal end of the probe; however, the proximal portion, the proximal end portion, or the proximal length of the probe need not include the proximal end of the probe. That is, unless context suggests otherwise, the proximal portion, the proximal end portion, or the proximal length of the probe is not a terminal portion or terminal length of the probe.

With respect to “distal,” a “distal portion” or a “distal end portion” of, for example, a probe disclosed herein includes a portion of the probe intended to be near or in a patient when the probe is used on the patient. Likewise, a “distal length” of, for example, the probe includes a length of the probe intended to be near or in the patient when the probe is used on the patient. A “distal end” of, for example, the probe includes an end of the probe intended to be near or in the patient when the probe is used on the patient. The distal portion, the distal end portion, or the distal length of the probe can include the distal end of the probe; however, the distal portion, the distal end portion, or the distal length of the probe need not include the distal end of the probe. That is, unless context suggests otherwise, the distal portion, the distal end portion, or the distal length of the probe is not a terminal portion or terminal length of the probe.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

The effect of temperature variations on the human body has been well documented. Elevated temperatures may be harmful to the brain under normal conditions, and even more importantly, during periods of physical stress, such as illness or surgery. Conversely, lower body temperatures, or mild hypothermia, may offer some degree of neuroprotection. Moderate to profound hypothermia (below 32° C.) tends to be more harmful to the body and may lead to death.

Targeted Temperature Management (TTM) refers to cooling or heating a patient to provide medical benefits, such as neuroprotection following a stroke or surgery. TTM or thermoregulation can be viewed in two different ways. The first aspect of temperature management includes treating abnormal body temperatures, i.e. cooling the body from elevated temperatures (hyperthermia), or warming the body to manage hypothermia. Hypothermia may occur in response to exposure to cold environments, trauma, or long complex surgical procedures. Hyperthermia may occur in response to systemic inflammation, sepsis, stroke, or other brain injury.

The second aspect of thermoregulation is a treatment that employs techniques that physically control a patient's temperature to provide a physiological benefit, such as cooling for a degree of neuroprotection. Studies have shown that treatment with mild hypothermia, defined as lowering core body temperature 2-3° C., confers neuroprotection in stroke victims, and may hasten neurologic recovery and improve outcomes when applied for 24 to 72 hours in cases of traumatic brain injury. In particular, research suggests that brain damage from a stroke may take hours to reach maximum effect. Neurologic damage may be limited and the stroke victim's outcome improved if a neuroprotectant therapy, such as cooling, is applied within this time frame.

A TTM system using medical pads can regulate body temperature for patients who undergo procedures requiring therapeutic TTM and/or to assist in controlling temperature for specific medical or surgical conditions. Such a system is described in U.S. Pat. No. 6,645,232, filed Oct. 11, 2001, and titled “Patient Temperature Control System with Fluid Pressure Maintenance,” and the medical pads are described in U.S. Pat. No. 6,375,674, filed Jan. 3, 2000, and titled “Cooling/Heating Pad and System,” both of which are incorporated herein by reference.

One problem that often arises with targeted temperature management (TTM) systems is applying medical pads to accommodate patients of different sizes effectively and comfortably. An ill-fitting medical pad may hinder effective transmission of thermal energy between the pad and the patient, while also giving rise to patient discomfort. For example, if the TTM pad is oversized, covering too much of a patient's body surface, the patient may be heated or cooled too strongly by the TTM pad. In another example, an undersized TTM pad may not heat or cool the patient sufficiently. As a result, typically, numerous TTM pad sizes are available, but selecting the correct size may be difficult and complex, as well as requiring multiple sizes to be stored. Moreover, there exists the high possibility of selecting the wrong size pad and making multiple kit components unusable, resulting in high waste of an expensive product and frustration on the part of users due to incorrect pad sizing. The disclosed embodiments of devices and methods can address this problem by adjusting the patient contact area of the thermal pad to better accommodate different patient sizes.

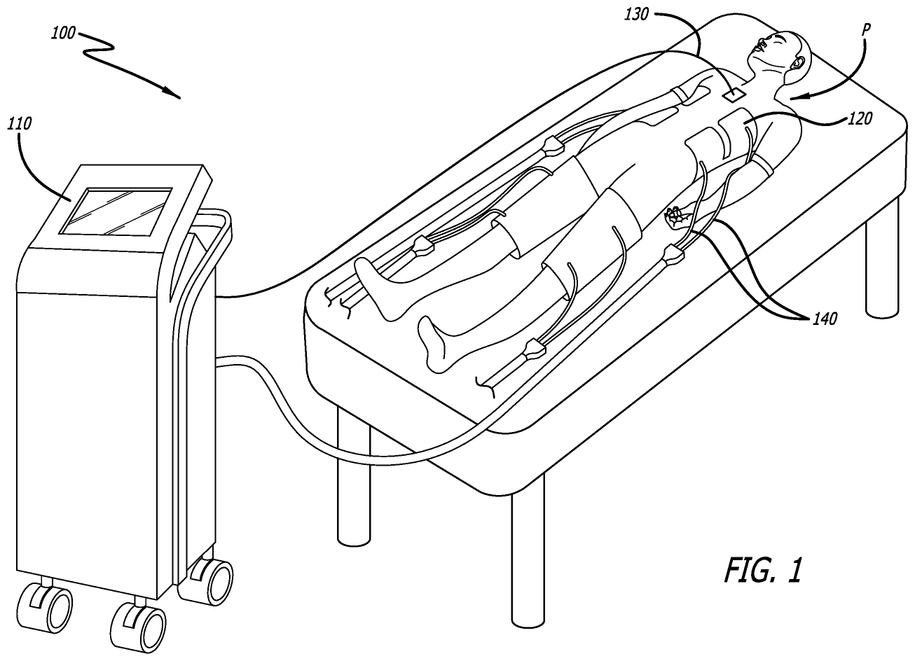

Reference is now made to FIG. 1, which illustrates a TTM system 100 using medical pads 120 for heating and/or cooling a patient P, according to some embodiments. The illustrated patient temperature control system 100 is a thermoregulatory system and apparatus that monitors and controls patient temperature within a range of 32° C. to 38.5° C. (89.6° F. to 101.3° F.). TTM system 100 is selectively interconnected to one or more medical contact pads 120 for exchanging thermal energy with patient P, and can also include a circulating pump for drawing temperature-controlled fluid (e.g., water or a gas) through pads 120 under negative pressure.

In some embodiments, TTM system 100 can include a control module 110, one or more disposable medical contact pads 120, a remote display in control module 110, a patient temperature probe 130, one or more fluid circulation lines 140, such as inlet and outlet lines to and from pads 120, and any additional accessories. In a typical embodiment, there may be two pads 120 placed on the patient's upper body as shown, and two on the patient's lower body. The TTM system 100 uses negative pressure to draw temperature-controlled fluid, such as water ranging between 4° C. and 42° C. (39.2° F. and 107.6° F.), through the pads 120 at approximately 0.7 liters per minute per pad. This results in heat exchange between the circulating fluid and the patient P. The patient temperature probe 130 is connected to the control module 110, and provides patient temperature feedback information to an internal control algorithm of control module 110. Based on such an internal control algorithm, control module 110 can increase or decrease the circulating water temperature so as to heat or cool patient P to a target patient temperature, which can be set by the clinician.

Fluid circulation lines 140 may include opposing tubing assemblies for interconnection to outlet and inlet ports of the circulating pump, with pads 120 fluidly interconnectable by means of opposing pad manifolds. FIG. 1 also illustrates the interconnection of one or more external patient temperature sensors 130 with a signal conditioning interface of control module 110. The temperature information received from external temperature sensors 130 may be utilized at a processor of control module 110 to determine the amount and rate of thermal exchange to be affected by system 100 in relation to the preset or user-defined patient target temperature. Accordingly, the processor may provide appropriate control drive signals to a heater, radiator/fan and/or auxiliary pump of TTM system 100. In an embodiment, the circulating pump, heater, radiator/fan, and/or auxiliary pump may be housed within control module 110.

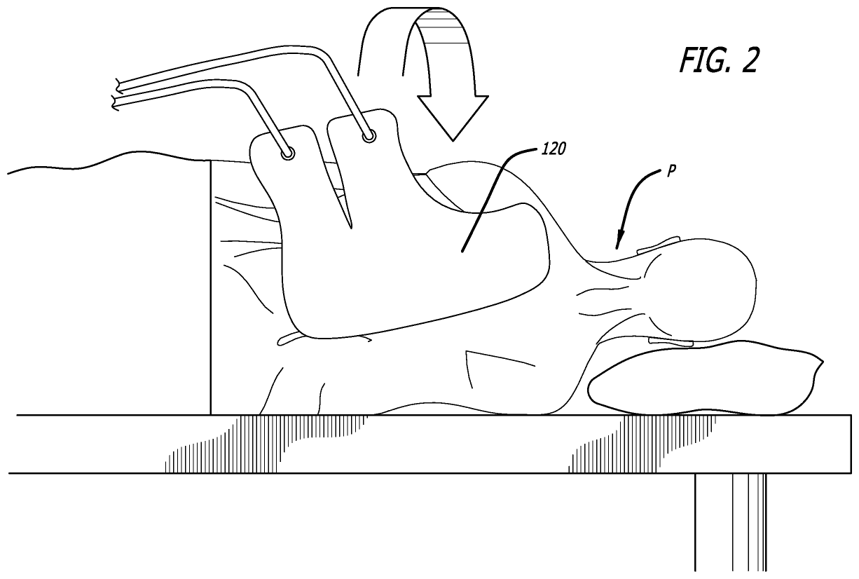

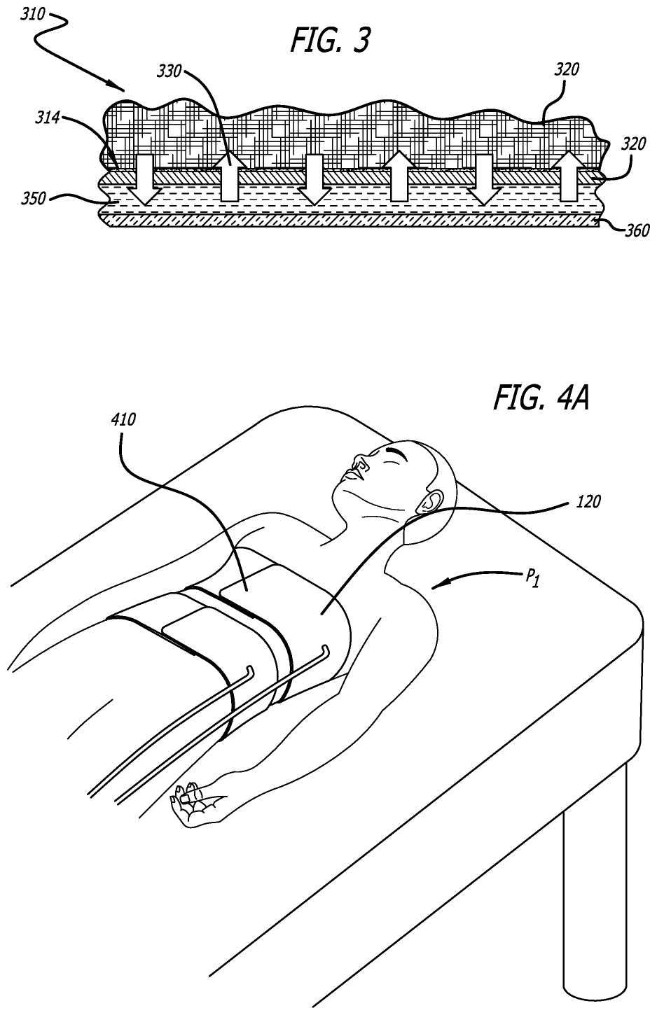

FIG. 2 illustrates a TTM medical pad 120 being placed on a patient P, according to some embodiments. Pad 120, and particularly an inner layer of pad 120 containing biocompatible hydrogel, can conform to the patient's skin, and thereby provide good thermal contact with patient P. The medical pad 120 can include several layers: an inner biocompatible hydrogel layer that adheres and conforms to the patient P, a fluid containing layer, one or more thin film layers which serve as a fluid barrier, and an outer insulating layer which prevents heat transfer to the environment (see FIG. 3). The hydrogel layer can have sufficient adhesive strength to hold pads 120 in place on patient P during the TTM therapy, yet not cause tissue damage when subsequently removed.

The pads may be available in extra-small, small, medium, and large sizes, as well as a universal pad. The clinician can determine the style, size, and number of pads 120 to be applied to patient P based on the patient procedure, application, or the available body surface area on patient P. For example, the clinician may place two pads 120 on the patient's upper body, such as on the patient's back and torso as illustrated in FIG. 2, and two pads on the patient's lower body, for example wrapped around the patient's thighs. The medical pads 120 will provide the best performance when the maximum number and correct size are used.

Due to the negative fluid pressure applied by system 100, significant fluid leakage will not occur, even if pads 120 are damaged or broken while fluid is flowing. Accordingly, pads 120 can be applied to the patient while fluid is already flowing through the pads. Depending on the objective of the treatment and the patient's level of arousal, pads 120 may be pre-warmed or pre-cooled prior to placement.

In order to place TTM pad 120, a clinician will first align the top of a first upper body pad 120 with axilla of the patient's outstretched arm. The clinician will then place the long side of pad 120 along the side of the patient's spine. Next, the clinician can wrap pad 120 from back to front as illustrated, ensuring that the pad's fluid inlet and outlet lines are lying anteriorly. For the lower body, the clinician can align the first lower body pad's lines with the knee and point downward. The clinician will wrap the long end of the first lower body pad laterally, and overlap medially if needed.

The clinician may then turn the patient P and place a second upper body pad on the patient's other side, leaving a space along the patient's spine. Next, the clinician can wrap a second lower body pad around the patient's other leg, ensuring that the shorter edge is placed medially and the longer side is wrapped laterally. Finally, if additional surface coverage is needed, the clinician can optionally place a universal medical pad on the patient's abdomen.

The medical pads 120 have inlet and outlet lines for the fluid flow, referred to herein as pad lines (see FIG. 1). These lines are connected to the pads 120 by means of a pad manifold. In particular, a Y-shaped fluid delivery line (FDL) contains one-way valves that connect to pad line connectors (e.g., a total of six connectors). Each side of the fluid delivery line can be placed by the patient's feet or along the patient's lower legs. The connectors can accommodate a full set of four pads 120 plus a maximum of two optional universal medical pads for larger patients. While holding the pad line tubing, the clinician can insert a pad line connector into the pad fluid delivery line manifold. For example, the clinician can push a respective connector toward the manifold to release associated catches, and then pull apart. Subsequently, the clinician can disconnect the lines, e.g., by squeezing wings on the connector together.

FIG. 3 illustrates a structure of an exemplary medical pad, according to some embodiments. TTM medical pad 120 comprises inner biocompatible hydrogel layer 340, which is a conformable, thermally conductive layer that can adjoin and conform to patient's skin 320. Further, the pad 120 may include an adhesive layer 341 disposed on the skin contacting side of the hydrogel layer 340 for adhering the pad 120 to the patient's skin 320. While not shown, a removable release liner may be provided over the adhesive surface 341 to protect the adhesive surface 341 from contamination while the pad 120 is not in use.

Pad 120 additionally comprises fluid containing layer 350 and insulation layer 360 for preventing loss of thermal energy to the environment. The fluid containing layer 350 can be defined between one or more film layers and/or insulation layer 360. The fluid can be heated or cooled to a temperature between 4° C. and 42° C. (39.2° F. and 107.6° F.), and can circulate through fluid containing layer 350, exchanging thermal energy 330 with patient's skin 320 via hydrogel layer 340, so as to warm or cool patient P to the target temperature. Although in this example, thermal energy 330 is shown flowing from skin 320 to the fluid in layer 350, heat 330 can flow in either direction between patient P and layer 350, so as to heat or cool patient P to the target temperature.

Alternatively, in some embodiments, pad 120 comprises hydrogel layer 340, a thin film layer which serves as a fluid barrier, and outer insulating layer 360 comprising foam with water channels.

A hydrogel is an appropriate material for layer 340 because the hydrogel is biocompatible, its adhesive strength does not tend to increase over time as compared with traditional adhesive, it tends to envelop hair on patient's skin 320, thereby facilitating good thermal contact, and its high water content results in relatively high thermal conductivity. Accordingly, hydrogel layer 340 may function as a thermally conductive layer, while also having sufficient adhesive properties so as to integrally provide an adhesive surface. Alternatively, in some embodiments, the conformable, thermally conductive layer and adhesive surface can be comprised of different materials. For example, an appropriate adhesive material may be sprayed or otherwise applied onto the surface of a layer of an appropriate conformable, thermally conductive material different than the adhesive material.

Fluid containing layer 350 can include tortuous fluid flow paths, which can be defined by dimples or other elongated members on insulation layer 360 or within the fluid containing layer 350. Such tortuous fluid flow paths can serve to regulate the fluid flow, and to inhibit the formation of boundary layers wherein some of the fluid remains substantially stationary along the inside surfaces of the fluid containing layer 350. Such boundary layers could reduce the efficiency of the pad 120 because the stationary fluid remains within the fluid containing layer 350, but eventually becomes ineffective at heating or cooling patient P as it approaches the existing temperature of patient P. Furthermore, the crisscrossed geometry of elongated members defining the tortuous flow paths also facilitates an even, low pressure drop between the inlet and the outlet required by a negative flow pressure circulating system.

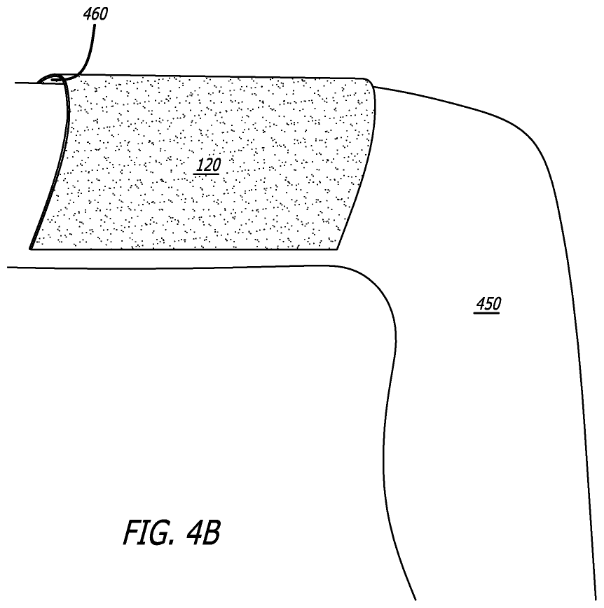

One need that frequently arises with targeted temperature management (TTM) is for the TTM medical pads to accommodate different patient sizes. An inadequately-fitting medical pad, such as that shown in FIG. 4A, may hinder effective transmission of thermal energy between the pad and the patient, and may also give rise to patient discomfort. In addition, the TTM pads may be inflexible, and cannot conform to variations in patient anatomy, as shown in FIG. 4B. Likewise, although universal TTM pads are designed to supplement coverage on arbitrary areas of a patient's body, they are not specifically designed to cover a particular area, such as a patient's torso, back, or legs, and moreover may not conform to the contours of these body parts. Disclosed herein are embodiments of TTM medical pads and methods for adjusting the patient contact area to better accommodate patients of different sizes, conforming to contours of the patient's anatomy, and cushioning the patient's skin from rough edges of the pads.

FIG. 4A shows a patient Pi with an oversized medical pad 120. In this example, as pad 120 is too large for patient Pi, portions 410 of pad 120 entirely cover the chest of patient Pi, which may run counter to the intention of the clinician overseeing the TTM therapy.

In addition to causing patient discomfort, such a situation could lead to energy waste, as well as ineffective temperature management. Because portions 410 of pad 120 overlap one another, and do not directly contact patient Pi, some of the heating and cooling power of pad 120 goes to waste. For example, since the TTM fluid flowing through pad 120 is at a substantially uniform temperature, net heat is not expected to be exchanged between the overlapping portions of pad 120. Instead, the excess flow of TTM fluid may heat or cool the ambient air around patient Pi. In another example, because portions 410 of pad 120 cover too much body surface of patient Pi, patient Pi may be heated or cooled too strongly by pad 120. In an acute case, overheating or overcooling patient Pi could potentially engender a risk of medical complications, particularly if patient Pi is in a vulnerable state, such as recovering from a stroke, from another medical emergency, or from a surgery. Thus, there is a need for a method to adjust the size of pad 120 in order to improve the fit of pad 120.

In addition to having a fixed size, in some cases, pad 120 may retain its flat shape and be unable to conform to contours of the surface of the patient's skin 320, and/or variations among patients. In particular, pad 120 may include an insulation layer 360 (see FIG. 3), such as foam. This insulation layer may be inflexible. As a result, pad 120 may be inflexible, and may not conform well to variations in patient anatomy.

FIG. 4B shows a TTM medical pad inadequately conforming to a contour of a patient's anatomy. In this example, pad 120, which contains an inflexible insulation layer, is placed on a patient body part 450, such as a leg of patient P. However, pad 120 does not conform fully to the contours of leg 450. In particular, while pad 120 can bend or curve over a long length scale, i.e. with a large radius of curvature, it may not be flexible enough to curve over shorter length scales. For example, pad 120 may curve sufficiently to drape over the patient's torso, as shown in FIGS. 2 and 4A, or over the patient's leg, as in the present example. But while pad 120 may curve over the girth of leg 450 in this example, it may do so with a larger radius of curvature than the contour of leg 450 itself. This can result in “bubbles” or gaps between pad 120 and leg 450, such as gap 460, as shown.

Such inflexibility can result in an effective thermal contact area smaller than the full pad area, that is, only part of the area of pad 120 effectively exchanges thermal energy with leg 450. For example, the portion of pad 120 curving about gap 460 may not contribute to the thermal contact with the patient's leg 450. Instead, similar to the case of FIG. 4A, some of the TTM fluid flowing in the pad's fluid containing layer may heat or cool the ambient air around gap 460.

Moreover, such a situation may limit treatment effectiveness, since less than the pad's full capacity for thermal exchange with leg 450 is utilized, and therefore some thermal energy may go to waste, even while patient P is heated or cooled inadequately. In an acute case, overheating or overcooling patient P could engender a risk of medical complications, particularly if patient P is in a vulnerable state. Accordingly, there is a need for a method to conform the shape of pad 120 in order to improve its fit to contours of the patient's anatomy. The disclosed embodiments can address these problems by providing a TTM pad that can flex, stretch, and conform to an appropriate size and shape for the patient.

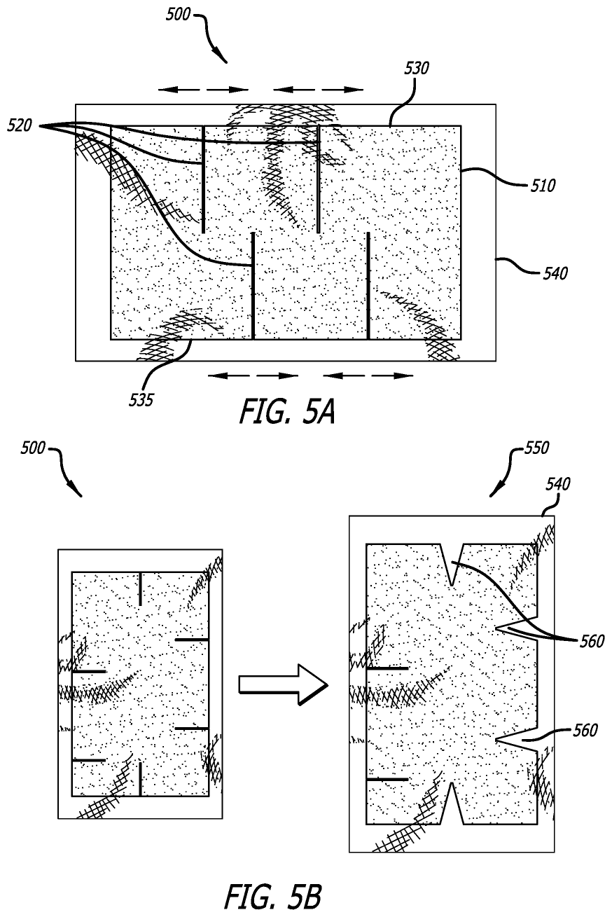

FIG. 5A illustrates a TTM medical pad 500 with slits and a flexible fabric cover, according to some embodiments. Like existing medical pads (see FIG. 3), pad 500 can include an insulation layer 510, such as foam. Foam layer 510 may be inflexible. Accordingly, in order to make pad 500 more flexible than existing medical pads, foam insulation layer 510 of pad 500 can include slits 520. Slits 520 can facilitate the stretching of pad 500 (see FIG. 5B). Slits 520 can also facilitate the pad 500 conforming to a contour of the patient's anatomy. In some embodiments, slits 520 may be in the insulation layer 510, thereby making the pad as a whole more flexible. In some embodiments, slits 520 are present in all the layers of pad 500. As should be understood, the pad 500 includes the same layers as the pad 120 discussed above that enable the pad 500 to exchange thermal energy with patient P.

In a typical example, the slits 520 may be oriented perpendicularly to the edges of pad 500. In the example of FIG. 5A, slits 520 are oriented vertically as shown, perpendicular to horizontal edges 530 and 535, which they also adjoin. The slits may also be oriented in other directions, such as at acute angles with the edges of pad 500, and are not limited by the present disclosure. In some instances, although not shown, a slit may be located within the interior of the pad 500 (e.g., not adjoining an edge of the pad 500).

In this example, slits 520 extend a distance along the vertical dimension (i.e., perpendicularly to edges 530 and 535). In particular, slits 520 extend slightly more than halfway across the width of pad 500 (i.e., vertical dimension as shown). Alternatively, the slits may extend exactly halfway across, or slightly less than halfway across. In some embodiments, the slits may extend a different distance across, such as 10%, 20%, 30%, 40%, 50%, 60%, 70%, or 80% across the pad, and are not limited by the present disclosure.

Because slits 520 extend a significant distance across the width of pad 500, slits 520 can facilitate the stretching of pad 500. In particular, if slits 520 are stretched with some opening angle α, such as 5°, 15°, 30°, 45°, or more, the result will be a relatively large extension in the length of edges 530 and 535. In particular, if L is the length of a respective slit of slits 520, then the length of edges 530 and 535 will increase by an amount approximately equal to L tan α. Accordingly, for a given opening angle α, the amount of stretching of edges 530 and 535 increases with L.

Likewise, slits 520 can facilitate the bending or curving of the plane of pad 500, and accordingly the TTM pad 500 can more readily conform to the contours of the patient's anatomy. In particular, because slits 520 divide pad 500 into narrower segments or strips, less force is required in order to bend or curve some or all of these segments. As a result, pad 500 can stretch, bend, or curve more easily, and can hence better conform to the contours of the patient's anatomy. This can result in better thermal contact, as a greater proportion of the full pad area can contact the patient, thereby improving TTM treatment effectiveness. Thus, flexible TTM pad 500 permits better thermal contact for the available pad area and alleviates gaps, “air pockets,” or “bubbles,” such as gap 460 of the example of FIG. 4B.

In addition, pad 500 includes flexible fabric cover 540, which further facilitates the stretching of the pad, as well as its ability to curve and conform to contours of the patient. For example, flexible fabric cover 540 may comprise one or more of nylon, polychloroprene, elastane, latex, isoprene, polyisoprene, elastolefin, polybutadiene, nitrile rubber, butyl rubber, a loose-woven fabric or other non-woven fabric (e.g., polypropylene, polyester, a blend of polypropylene and polyester, sintered foam (e.g., titanium, aluminum, stainless steel, etc.) or molded pulp (molded fiber), which is often made from recycled paperboard and/or newsprint). In some embodiments, the cover 540 can include a combination of materials, for example both an elastic material, such as latex, and a fabric, such as cotton. For example, the cover 540 may comprise a loose-woven fabric with some latex, nylon, or polychloroprene threads in the weave. In another example, cover 540 may comprise a loose-woven fabric with one or more elastic bands that impart elasticity to the cover. In yet another example, cover 540 can comprise a loose-woven fabric with both elastic threads and elastic bands.

In addition, the flexible fabric cover 540 may have a soft texture configured to reduce pressure on skin of the patient from an edge or surface of the pad. For example, the cover 540 can comprise a soft fabric, such as cotton, which can soften the edges of the TTM pad making contact with the patient's skin. Existing TTM pads have edges that may be uncomfortable and cause skin damage due to chafing, depending on the contacting anatomy. Specifically, the medical pads may have a harsh edge that may cause discomfort and irritation for some patients. Even though the pads can contain a pliable material, like a hydrogel, that can conform to the patient's skin and provide good thermal contact, some patients may experience skin irritation. In some cases, patients may use the pads for extended periods, exacerbating such discomfort and irritation after repeated contact. Fabric 540 may help form a softer, more comfortable edge. In some embodiments, fabric cover 540 has properties to reduce chafing or skin irritation compared with conventional TTM pads. For example, fabric 540 may be softer, smoother, or less harsh than the hydrogel layer, or may be treated with a skin lotion, such as a hydrating lotion.

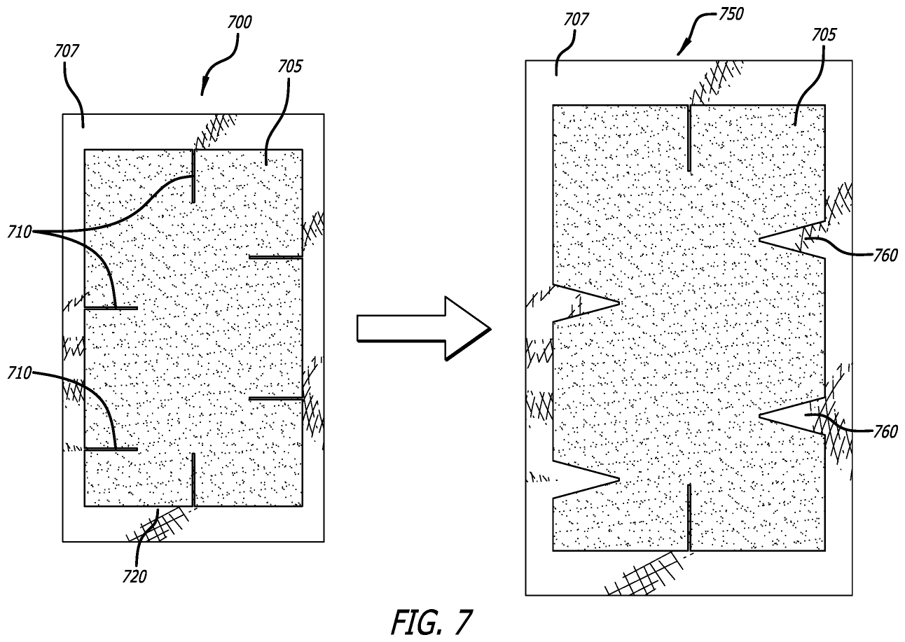

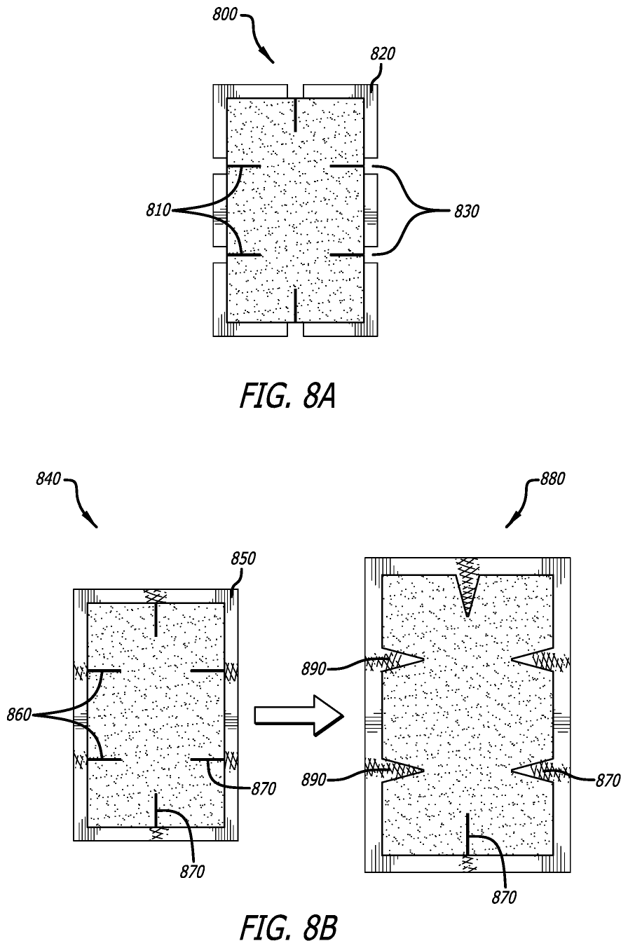

In some embodiments, flexible fabric cover 540 can completely cover or surround pad 500, whereas in other embodiments, the cover may only cover part of pad 500, such as the edges of pad 500 and/or portions of the pad surface that coincide with slits 520. For example, the flexible fabric cover can comprise an exterior border portion surrounding the pad's perimeter, and a slit-covering portion coinciding with slits 520 (see FIG. 7). In another example, the flexible fabric cover can coincide with the slits 520, while an edge guard surrounds the pad's perimeter (see FIGS. 8A-8B). By covering the slits, fabric cover 540 may also protect TTM pad 500 from cracking or tearing, e.g., by preventing the slits from being flexed too far.

FIG. 5B illustrates flexing of a TTM medical pad with slits and a flexible fabric cover, according to some embodiments. In this example, pad 500 is stretched to a flexed configuration 550. The slits are stretched with opening angles 560, as described in the example of FIG. 5A. Moreover, flexible cover 540 comprises flexible, elastic materials. Accordingly, flexed pad 550 can easily expand to provide TTM therapy to an area larger than that provided by the unflexed TTM pad 500, as shown.

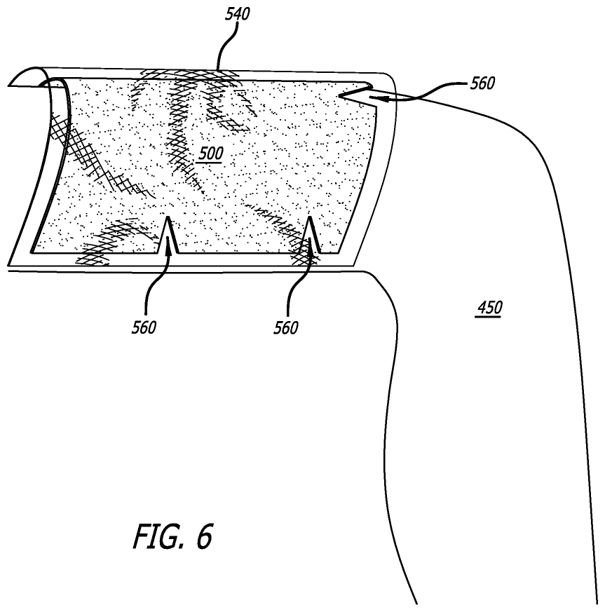

The flexibility illustrated in this example provides a number of advantages. First, the larger area of flexed pad 550 enables the same TTM pad to be used on patients of a range of sizes. As a result, a hospital or other facility can purchase and maintain fewer sizes of TTM pads, but can still access pads capable of fitting patients properly when needed. Likewise, the disclosed flexible TTM pad can simplify a clinician's process of selecting a pad size appropriate for a given patient. In particular, due to the expansibility of the disclosed pads, fewer sizes of TTM pads are needed, so selecting the correct size of TTM pad to fit a patient is simpler, faster, and less confusing. In addition, by making it possible to adjust the patient contact area and reducing the number of different sized pads needed, flexing the TTM pad 550 reduces the likelihood a clinician will select a pad of the wrong size, which could make multiple kit components unusable. Thus, the disclosed TTM pad can also reduce the risks of waste, clinician frustration, and medical complications due to incorrect pad sizing. Finally, flexed pad 550 can conform to a contour of a patient's anatomy, as illustrated in FIG. 6.

FIG. 6 shows the TTM medical pad of FIG. 5B conforming to a contour of a patient's anatomy, according to some embodiments. In some embodiments, the presence of slits 520 can facilitate bending or curving of the plane of pad 500. In this example, similar to FIGS. 5A-5B, the slits are stretched with opening angles 560, and pad 500 is flexed.

Accordingly, the disclosed TTM pad 500 can conform to the contours of the patient's anatomy, such as to leg 450, more readily than a conventional TTM pad with an inflexible insulating layer. In this example, pad 500 can curve over the girth of leg 450, with a small radius of curvature, matching the contour of leg 450. As a result, pad 500 fits snugly on leg 450 without gaps, “air pockets,” or “bubbles,” such as gap 460 in the example of FIG. 4B. In addition, fabric 540 is flexible, and therefore can also facilitate the pad's ability to stretch, bend, and curve to conform to leg 450.

The improved fit of pad 500 can improve thermal contact compared with the situation in FIG. 4B, enabling the full area of pad 500 to exchange thermal energy with leg 450. Accordingly, in this example, treatment effectiveness may be significantly improved, since the pad's full patient contact surface is utilized for thermal exchange with leg 450, and therefore patient P can be heated or cooled more quickly, and with less energy waste. Moreover, the risks of improper thermal contact, sub-optimal heating or cooling, and medical complications are reduced.

FIG. 7 illustrates a TTM medical pad 700 including an insulating layer 705 and a flexible fabric cover 707 surrounding its perimeter 720 and covering slits 710, according to some embodiments. In an embodiment, the fabric cover 707 can protect the patient's skin from discomfort and irritation due to pressure from edges of the TTM medical pads. In this example, the fabric cover 707 surrounds perimeter 720 in order to protect the patient from such edges.

Specifically, the medical pads may have a harsh edge, for example at curves or corners, that may cause discomfort and irritation for some patients. Even though the pads can contain an adaptable material, like a hydrogel, that can conform to the patient's skin and provide good thermal contact, some patients may still experience skin irritation while using the pads. In some cases, patients may use the pads for extended periods, exacerbating such discomfort and irritation after repeated contact. Embodiments of the disclosed apparatus and system can use a soft fabric cover 707, as in this example, or an edge guard (see FIGS. 8A-8B) to address this problem. In this example, when pad 700 is placed on the patient, the soft texture of the flexible fabric cover 707 can dissipate pressure from any edges surrounding perimeter 720 of pad 700, thereby improving comfort and protecting the patient's skin.

In addition, the slits 710 can also be covered by fabric. For example, the fabric may be affixed to the edges of the insulating layer 705 forming the slits 710 such that the fabric serves as a webbing in the opening formed when the pad 700 is in a flexed configuration 750. In this example, pad 700 is shown stretched to the flexed configuration 750. The slits are stretched with opening angles 760, as described in the example of FIG. 5A. In this embodiment, the fabric cover 707 can also protect the patient's skin from harsh edges within slits 710, particularly in the case where slits are present in all layers of pad 700, including the hydrogel layer that makes contact with the patient's skin. By covering the slits, the fabric cover 707 may also protect TTM pad 700 from cracking or tearing, e.g., by providing elastic resistance that prevents the slits from being flexed too far.

FIG. 8A illustrates a TTM medical pad 800 with an edge guard 820 with gaps 830 surrounding the pad's perimeter, and a flexible fabric covering slits 810, according to some embodiments. In this example, an edge guard 820 may surround some or all of the edges of pad 800, and extend outwardly from the edges. The edge guard can distribute pressure from any harsh edges of pad 800, for example around the pad's perimeter, thereby improving comfort and protecting the patient's skin. Edge guards are described in US Provisional patent application having Attorney Docket No. 101674.0393PRO, filed Jan. 27, 2021, and titled “Soft Border for Targeted Temperature Management,” and in US Provisional patent application having Attorney Docket No. 101674.0394PRO, filed Jan. 25, 2021, and titled “Cooling/Heating Medical Pad with Softened Edges,” both of which are incorporated herein by reference.

In order to reduce any irritation affecting the patient's skin 320 when in contact with TTM medical pad 800, in some embodiments edge guard 820 is comprised of a soft or pliant material such as silicone, silicone polydimethylsiloxane (PDMS), silicone rubber, or siloxane, serving as a shock-absorbing barrier between pad 800 and skin 320. In some embodiments, another material may be used, such as low-density polyethylene (LDPE), ethylene-vinyl acetate (EVA), expanded polypropylene, polyether block amide (PEBA), polystyrene, an elastomer, another plastic, reinforced foam, latex, or rubber. Edge guard 820 can distribute force from the pad 800 over a wider area, and can thereby reduce pressure on the patient's skin 320 and ameliorate side effects resulting from TTM treatment, such as patient discomfort and skin irritation.

Alternatively, the edge guard 820 can include an elastic outer covering shell comprising a material such as woven fabric, any of the fabric materials described in the example of FIG. 5A, plastic (such as polyvinyl chloride (PVC), polyethylene, or polyurethane), or latex. The elastic outer shell can enclose a soft filling, such as soft foam, cotton gauze, mesh, polyester, wool, or latex. Because the filling is soft and elastic, it can absorb mechanical pressure or shocks from the edge of pad 800 and cushion the patient's skin 320, thereby increasing patient comfort and improving patient tolerance of an extended TTM treatment. Moreover, edge guard 820 can act as a barrier between the edges of pad 800 and the patient's skin 320, i.e., it can prevent direct contact, rubbing, chafing, and the like between the edge of pad 800 and skin 320.

As shown, edge guard 820 can extend from the edges of pad 800, absorbing and distributing forces from the hydrogel layer and/or the other layers of pad 800. Because edge guard 820 is pliant, it may conform to the patient's skin. Edge guard 820 can have gaps 830, which can enable the pad 800 to flex and stretch, similar to the function of slits 810 in the pad itself. In some cases, gaps 830 can coincide with the position of slits 810. In an embodiment, the gaps can be similar in design to slits 810, i.e., they may open with opening angles that facilitate the flexing of pad 800, but may be larger than the slits 810. Alternatively, in some embodiments, the gaps may be significantly larger, or the edge guard may only surround a portion of the perimeter. For example, the edge guard may be situated at a corner, bend, or curve of medical pad 800, where the pad may have a particularly harsh or rough-textured edge.

FIG. 8B illustrates a TTM medical pad 840 with an edge guard 850 with slits 860 surrounding its perimeter, and a flexible fabric 870 covering the slits, according to some embodiments. In this example, an edge guard 850 may surround some or all of the edges of pad 840, and extend outwardly from the edges. The edge guard can distribute pressure from any harsh edges of pad 800, for example around the pad's perimeter, thereby improving comfort and protecting the patient's skin.

In some embodiments, edge guard 850 is comprised of a soft or pliant material such as silicone, silicone polydimethylsiloxane (PDMS), silicone rubber, or siloxane, serving as a shock-absorbing barrier between pad 840 and skin 320. Alternatively, the edge guard 820 can include an elastic outer covering shell comprising a material such as woven fabric, any of the fabric materials described in the example of FIG. 5A, plastic, or latex. The elastic outer shell can enclose a soft filling, such as soft foam, cotton gauze, mesh, polyester, wool, or latex.

As shown, edge guard 850 can extend from the edges of pad 840, absorbing and distributing forces from the hydrogel layer and/or the other layers of pad 840. Because edge guard 850 is pliant, it may conform to the patient's skin. In addition, edge guard 850 can have slits 860, which can coincide with the position of the slits in pad 840, and enable the edge guard to flex and stretch along with pad 840. In some embodiments, edge guard 850 may only surround a portion of the perimeter, for example, a corner, bend, or curve of medical pad 840. In this case, the edge guard may still have slits 860, for example coinciding with the slits in the portion of pad 840 that is covered by edge guard 850, and/or spaced regularly. Alternatively, edge guard 850 may completely surround pad 840, but may include slits 860 to facilitate flexing.

In addition, the slits 860 can also be covered by fabric. In this example, pad 840 is shown stretched to its flexed configuration 880. The flexed slits 890 are stretched with opening angles, as described in the example of FIG. 5A. In this embodiment, the fabric cover 870 can also cover the slits, protecting the patient's skin from harsh edges within flexed slits 890, particularly in the case where slits are present in all layers of pad 840, including the hydrogel layer that makes contact with the patient's skin. By covering the slits, fabric cover 870 can also protect TTM pad 840 from cracking or tearing, e.g., by providing elastic resistance that prevents the slits from being flexed too far.

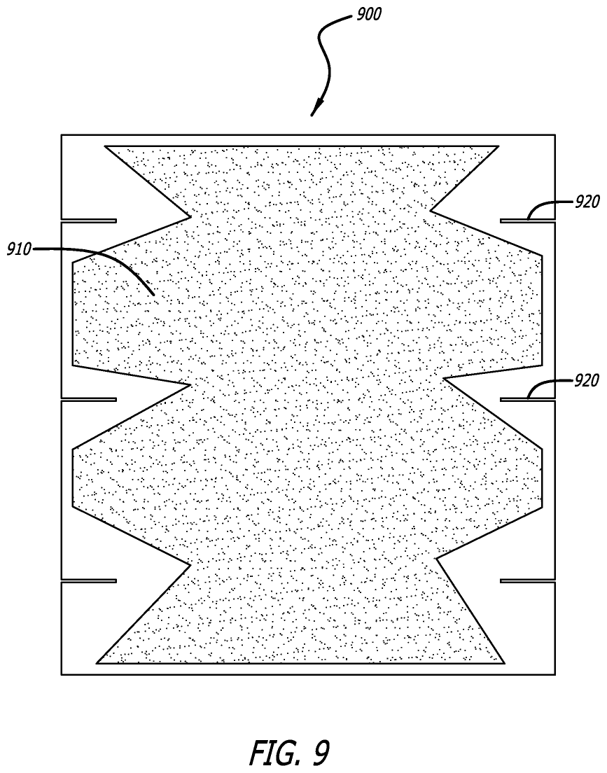

FIG. 9 illustrates a TTM medical pad 900 with a fluid containing layer 910 and slits 920, arranged so that the slits 920 do not coincide with the fluid containing layer 910, according to some embodiments. As described in the example of FIG. 3, fluid containing layer 910 may include a plurality of tortuous fluid flow paths. In some embodiments, the one or more slits 920 are located in parts of the insulating layer, and/or the other layers of pad 900, that do not coincide with the fluid containing layer, or with the tortuous fluid flow paths of the fluid containing layer. As should be understood, inclusion of the slits 920 facilitates the patient contact surface conforming to a contour of a body part of the patient.

In particular, in some embodiments, the pad flow pattern may be modified to accommodate the slit locations, so the slits can be located away from the fluid flow paths within the fluid containing layer 910. That is, the fluid containing layer may be shaped, oriented, and/or located in regions of pad 900 that do not coincide with slits 920. In various embodiments, different patterns may be chosen for the fluid containing layer that result in free, well-circulated TTM fluid flow, while also accommodating the slits. For example, the fluid containing layer may be restricted to an interior area of the pad 900, while the slits 920 may be located at the pad's edges. Alternatively, the fluid containing layer 910 may be arranged so as to circumvent the slits 920.

In this example, fluid containing layer 910 is arranged such that slits 920 do not coincide with any fluid flow paths. In particular, fluid containing layer 910 is arranged to circumvent slits 920 by having a shorter radial extent in directions where slits 920 are present at the edge of pad 900. Moreover, fluid containing layer 910 has a longer radial extent in directions where no slits are present at the edge of the pad, so as to provide more area for the TTM fluid flow and a more effective thermal energy transfer with the patient. As a result of this arrangement of fluid containing layer 910, the TTM fluid flows freely through fluid containing layer 910, without any interference from slits 920.

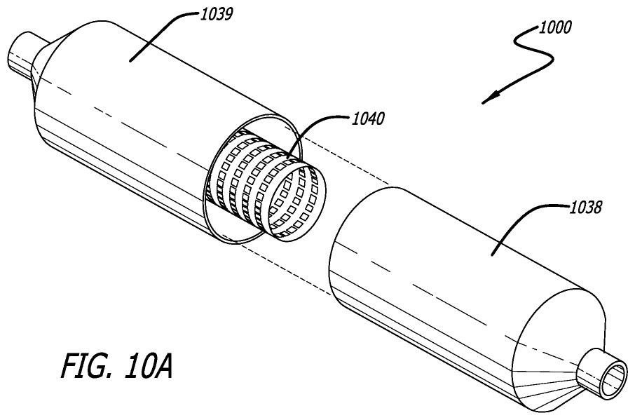

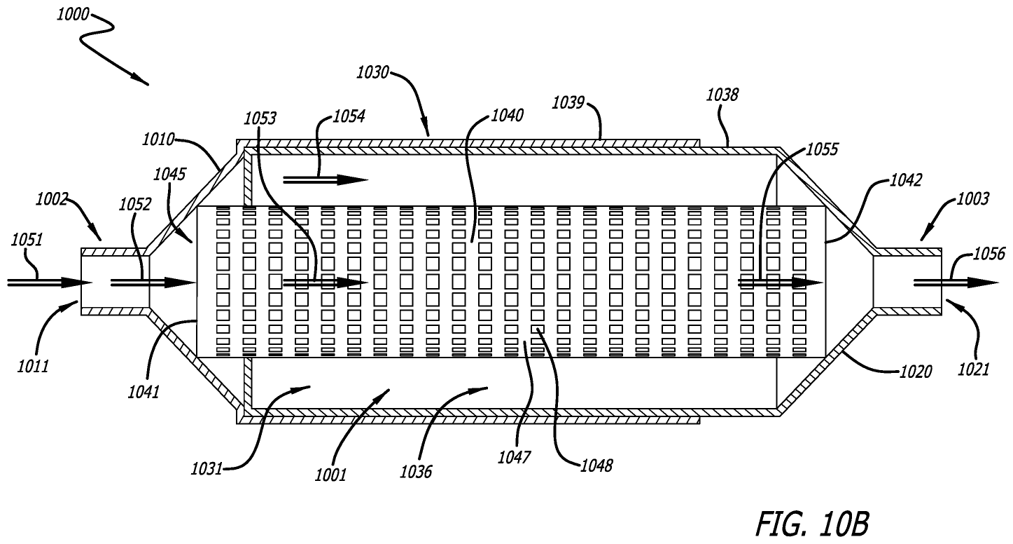

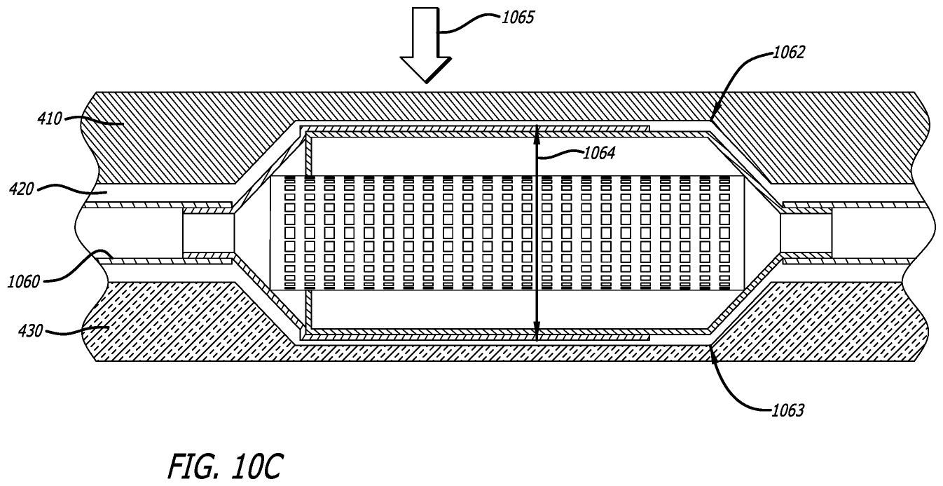

Referring now to FIGS. 10A-10B, a filter 1000 is illustrated that may be included with the TTM system 100, in accordance with some embodiments. The filter 1000 may be disposed in line with a TTM fluid flow path of the TTM system 100 so that the circulating TTM fluid flows through the filter 1000. The filter 1000 may be configured to remove (i.e., filter out) material/particles having a size of 0.2 microns or larger from the TTM fluid without causing a flow restriction of the fluid.

The filter 1000 comprises a longitudinal shape having a flow path 1001 extending from a first end 1002 to a second end 1003. The filter 1000 comprises a diffuser 1010 adjacent the first end 1002, a nozzle adjacent 1020 the second end 1003, and a body 1030 extending between the diffuser 1010 and the nozzle 1020. Along the diffuser 1010, a cross-sectional flow area of the filter 1000 expands from an inlet flow area 1011 to a body flow area 1031 and along the nozzle 1020, the cross-sectional flow area of the filter 1000 contracts from the body flow area 1031 to an outlet flow area 1021. In some embodiments, the inlet flow area 1011 and the outlet flow area 1021 may be substantially equal.

In some embodiments, the body flow area 1031 may be constant along the body 1030. In other embodiments, the body flow area 1031 may vary along a length of the body 1030 such that the body flow area 1031 is greater or less along middle portion of the body 1030 than at the ends of the body 1030. In some embodiments, the body flow area 1031 may be circular.

The filter 1000 comprises an inner tube 1040 disposed within the body 1030 extending along the length of body 1030. The inner tube 1040 may be coupled to the diffuser 1010 at a first inner tube end 1041 so that fluid entering the filter 1000 at the first end 1002 also enters the inner tube 1040 at the first inner tube end 1041. The inner tube 1040 may be coupled to the nozzle 1020 at a second inner tube end 1042 so that fluid exiting the filter 1000 at the second end 1003 also exits the inner tube 1040 at the second inner tube end 1042.

The inner tube 1040 comprises an inner tube flow area 1045 extending the length of the inner tube 1040. The inner tube flow area 1045 may be greater than the inlet flow area 1011 and/or the outlet flow area 1021. The inner tube flow area 1045 may be constant along the length of the inner tube 1040. In some embodiments, the inner tube flow area 1045 may vary along the length of the inner tube 1040. In some embodiments, the inner tube 1040 may comprise a circular cross section. The inner tube 1040 and the body 1030 may be configured so that the body flow area 1031 comprises a combination of the inner tube flow area 1045 and an annular flow area 1036.

The inner tube 1040 comprises a porous a circumferential wall 1047. The porous wall 1047 may be configured so that fluid may flow through the porous wall 1047, i.e., through the pores 1048 of the porous wall 1047. Consequently, fluid may flow through the porous wall 1047 from the inner tube flow area 1045 to the annular flow area 1036 and from the annular flow area 1036 into the inner tube flow area 1045.

In use, the longitudinal velocity of the fluid may change along the length of the filter 1000. As the volumetric fluid flow through the filter is constant, the longitudinal velocity of the fluid may be at least partially defined by the flow areas of the filter 1000 as described below. The fluid may enter the filter 1000 at a first longitudinal velocity 1051 and decrease along the diffuser so that the fluid enters the inner tube at a second velocity 1052 less than the first longitudinal velocity 1051. At this point, a portion of the fluid may flow through the porous wall 1047 from the inner tube flow area 1045 into the annular flow area 1036 to divide the fluid flow into a third velocity 1053 within the inner tube flow area 1045 and a fourth velocity 1054 within the annular flow area 1036. The fourth velocity 1054 may be less than the third velocity 1053. A portion of the fluid may then flow back into the inner tube flow area 1045 from the annular flow area 1036 to define a fifth velocity 1055 along the inner tube flow area 1045 which may be about equal to the second velocity 1052. The fluid may then proceed along the nozzle 1020 to define a sixth velocity 1056 exiting the filter 1000. In some embodiments, the first velocity 1051 and the sixth velocity 1056 may be about equal.

The filter 1000 may be configured to remove harmful bacteria and viruses from the fluid using sedimentation principles. In use, the filter 1000 may be oriented horizontally so that the direction of fluid flow through the filter 1000 is perpendicular to a gravitational force 1065. In some instances, bacteria, viruses, and other particles within the fluid may have a greater density than the fluid and as such may be urged by the gravitational force 1065 (i.e., sink) in a direction perpendicular to the fluid flow direction. In some instances, particles within the inner tube flow area 1045 may sink toward and through the porous wall 1047 into the annular flow area 1036. Particles within the annular flow area 1036 may then sink toward an inside surface 1031 of the body 1030 and become trapped adjacent the inside surface 1031. The geometry of the filter 1000 may be configured to allow 0.2-micron bacteria/virus particles to fall out of the flow of TTM fluid and become trapped along the inside surface 1031.

In some embodiments, the filter 1000 may be configured so that flow of fluid from the inner tube flow area 1045 into the annual flow area 1036 my drag particles through the porous wall 1047. In some embodiments, the inlet flow area 1011, the inner tube flow area 1045, and the annual flow area 1036 may be sized so that the third velocity 1053 is less than about 50 percent, 25 percent, or 10 percent of the first velocity 1051 or less. In some embodiments, the body 1030 and the inner tube 1040 may be configured so that the fourth velocity 1054 is less than the third velocity 1053. In some embodiments, the fourth velocity 1054 may less than about 50 percent, 25 percent, or 10 percent of the third velocity 1053 or less.

In some embodiments, the filter 1000 may be configured so that the flow within the inner tube flow area 1045 is laminar flow, i.e., so that the velocity of the fluid flow adjacent to or in close proximity to an inside surface 1041 of the porous wall 1047 is less than the velocity at a location spaced away from the inside surface 1041. In such an embodiment, the particles may more readily sink toward and through the porous wall 1047.

In some embodiments, the filter 1000 may be configured so that the fluid flow within the annual flow area 1036 is laminar flow, i.e., so that the velocity of the fluid flow adjacent to or in close proximity to inside surface 1031 of the body 1030 is less than the velocity at a location spaced away from the inside surface 1031. In such an embodiment, the particles may more readily sink toward and be trapped along the inside surface 1031.

The filter 1000 may comprise three components including the inner tube 1040 an inner body shell 1038, and an outer body shell 1039. Each of the three components may be formed via the plastic injection molding process. Assembly of the filter 1000 may include capturing the inner tube 1040 within the inner body shell 1038 and the outer body shell 1039 and sliding the inner body shell 1038 into the outer body shell 1039 wherein the fit between the inner body shell 1038 and the outer body shell 1039 is an interference fit.

In some embodiments, the filter 1000 may be disposed within the pad 120. FIG. 10C shows a detail cross-sectional view of the pad 120 including the filter 1000 disposed within the fluid containing layer 350. The filter 1000 is coupled in line with an internal flow path 1060 within the fluid containing layer 350 so that fluid circulating within the pad 120 passes through the filter 1000. The filter 1000 may be sized so that the inlet flow area 1011 and the outlet flow area 1021 are similar to a cross-sectional flow area of the internal flow path 1060 within the fluid containing layer 350. The internal flow path 1060 may be comprised of tubing (e.g., similar to the fluid delivery lines 140) that is disposed within the fluid containing layer 350. In such embodiments, the tubing of the internal flow path 1060 receives the fluid from a fluid delivery line 140 at an inlet port. The fluid flows through the tubing of the internal flow path 1060, passing through the filter 1000, toward an outlet port, at which point the fluid exits the pad 120 and is received by a second fluid delivery line 140.

In some embodiments, a thickness of the fluid containing layer 350 may increase adjacent the filter 1000 to accommodate a body diameter 1064 of the filter 1000. To further accommodate the body diameter 1064, the insulation layer 360 and/or the thermal conduction layer 340 may comprise internal depressions 1062, 1063, respectively.

In some embodiments, one or more filters 1000 may be disposed in line with the flow of fluid at other locations of the TTM system 100. In some embodiments, one or more filters 1000 may be disposed within the TTM module 110. In some embodiments, one or more filters 1000 may be disposed in line with one or more of the fluid delivery lines 140. In some embodiments, the filter 1000 may be disposed in line with a fluid conduit of the pad external to the fluid containing layer 350 such as a conduit extending between a pad connector and the pad 120.

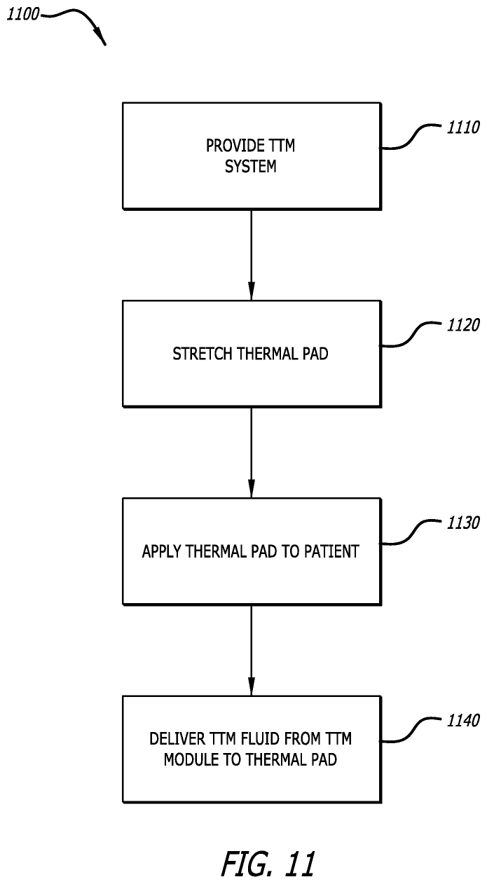

FIG. 11 illustrates a method 1100 of using a TTM medical pad with slits and a flexible fabric cover, according to some embodiments. Each block illustrated in FIG. 11 represents an operation performed in the method 1100 of using a TTM medical pad with slits and a flexible fabric cover. In various embodiments, the method can be performed by one or more users, such as nurses, doctors, or other clinicians, etc.

As an initial step in the method 1100, the user can provide a TTM system (block 1110) comprising a TTM module configured to provide a TTM fluid, a thermal pad configured to receive the TTM fluid from the TTM module to facilitate thermal energy transfer between the TTM fluid and a patient, and a fluid delivery line (FDL) extending between the TTM module and the thermal pad.

The pad comprises a fluid containing layer for containing and circulating the TTM fluid, and a patient contact surface to facilitate thermal energy exchange with the patient. In addition to its internal layers, the pad can have a patient contact surface. The patient contact surface can facilitate thermal energy exchange with the patient, that is, thermal energy can flow between the TTM fluid and the patient's skin through the patient contact surface. One or more slits are arranged in at least the insulating layer of the pad to facilitate stretching the pad. In some embodiments, the slits can also facilitate the patient contact surface conforming to a contour of a body part of the patient.

In some embodiments, the medical pad further comprises a flexible fabric cover, which also facilitates stretching the pad. In some embodiments, the flexible fabric cover has a soft texture configured to reduce pressure on skin of the patient from an edge or surface of the pad. In various embodiments, the fabric cover can cover the entire pad, surround the pad's perimeter, and/or cover the slits in the pad. In some embodiments, the pad can comprise an edge guard surrounding all or part of the pad's perimeter. In various embodiments, the slits are only present in the insulating layer, and/or are present in all layers of the pad, including the fluid containing layer. In some embodiments, the slits are arranged in portions of the insulating layer not coinciding with the fluid containing layer, or with the tortuous fluid flow paths in the fluid containing layer.

Next, the user can stretch the thermal pad (block 1120). The stretching can expand the patient contact surface from a first patient contact area to a larger second patient contact area. The ability to resize the pad provides advantages, such as enabling the pad to be adjusted to a patient's size, simplifying a clinician's process of selecting a pad size appropriate for a given patient, reducing the number of different pad sizes that must be maintained in stock, and reducing the risks of waste and medical complications due to incorrect pad sizing. In some embodiments, stretching or flexing the pad can also facilitate the patient contact surface conforming to a contour of a body part of the patient.

Next, the user can apply the thermal pad on the patient (block 1130). As described in the example of FIG. 2, the user can align the top of a first upper body pad with axilla of the patient's outstretched arm. The user can then place the pad along the side of the patient's spine. The user can wrap the pad from the patient's back to front. The user can align the first lower body pad's lines with the knee and point downward. The user can wrap the first lower body pad laterally, and overlap medially if needed. The user may then turn the patient and place a second upper body pad on the patient's other side, leaving a space along the patient's spine. The user can wrap a second lower body pad around the patient's other leg. Finally, if additional surface coverage is needed, the user can optionally place a universal medical pad on the patient's abdomen.

Finally, the user can configure the TTM system to deliver the TTM fluid from the TTM module to the thermal pad via the fluid delivery line (FDL) (block 1140). The TTM fluid can circulate through the fluid containing layer of the TTM pads, thereby heating or cooling the patient.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A medical pad for exchanging thermal energy between a targeted temperature management (TTM) fluid and a patient, the pad comprising:
an insulating layer;
a fluid containing layer for containing the TTM fluid, the fluid containing layer configured for circulating the TTM fluid within the fluid containing layer; and
a patient contact surface defining a first patient contact area to facilitate thermal energy exchange with the patient, wherein
one or more slits are arranged in at least the insulating layer of the pad to facilitate stretching the pad, wherein the stretching expands the patient contact surface from the first patient contact area to a larger second patient contact area.

2. The medical pad of claim 1, wherein one or more fluid containing layer slits are arranged in the fluid containing layer, the fluid containing layer slits coinciding with the one or more slits of the insulating layer.

3. The medical pad of claim 1, wherein the one or more slits facilitate conforming of the patient contact surface to a contour of a body part of the patient, wherein the patient contact surface contacts the body part.

4. The medical pad of claim 1, further comprising a flexible fabric cover, wherein the flexible fabric cover further facilitates the stretching of the pad.

5. The medical pad of claim 4, wherein the flexible fabric cover has a soft texture configured to reduce pressure on skin of the patient from an edge or surface of the pad.

6. The medical pad of claim 4, wherein the flexible fabric cover comprises one or more of nylon, polychloroprene, elastane, latex, isoprene, polyisoprene, elastolefin, polybutadiene, nitrile rubber, butyl rubber, or a loose-woven fabric.

7. The medical pad of claim 4, wherein the flexible fabric cover comprises:
an exterior border portion surrounding a perimeter of the pad, and
a slit-covering portion coinciding with the one or more slits in the insulating layer.

8. The medical pad of claim 4, wherein the flexible fabric cover facilitates conforming of the patient contact surface to a contour of a body part of the patient, wherein the patient contact surface contacts the body part.

9. The medical pad of claim 4, wherein:
the flexible fabric cover coincides with the one or more slits in the insulating layer, and
an edge guard surrounds a perimeter of the pad, the edge guard configured to reduce pressure from an edge or surface of the pad on skin of the patient.

10. The medical pad of claim 9, wherein the edge guard comprises one or more gaps coinciding with the one or more slits of the insulating layer.

11. The medical pad of claim 9, wherein the edge guard has one or more edge guard slits coinciding with the one or more slits of the insulating layer.

12. The medical pad of claim 9, wherein the edge guard includes:
a pliant edge guard cover, and
a cushion filling contained within the pliant edge guard cover.

13. The medical pad of claim 9, wherein the edge guard comprises silicone or another pliant shock-absorbent material.

14. The medical pad of claim 1, wherein:
the fluid containing layer includes a plurality of tortuous fluid flow paths, and
the one or more slits are arranged in portions of the insulating layer not coinciding with the fluid flow paths of the fluid containing layer.

15. A method of providing a targeted temperature management (TTM) therapy to a patient, comprising:
providing a TTM system including:
a TTM module configured to provide a TTM fluid,
a thermal pad configured to receive the TTM fluid from the TTM module to facilitate thermal energy transfer between the TTM fluid and a patient, and
a fluid delivery line (FDL) extending between the TTM module and the thermal pad, the FDL configured to provide TTM fluid flow between the TTM module and the thermal pad, wherein:
the thermal pad comprises an insulating layer and a patient contact surface defining a first patient contact area to facilitate thermal energy exchange with the patient, and
one or more slits are arranged in at least the insulating layer of the pad;
stretching the pad, wherein:
the stretching is facilitated by the one or more slits in the insulating layer, and
the stretching expands the patient contact surface from the first patient contact area to a larger second patient contact area;
applying the pad to the patient; and
delivering TTM fluid from the TTM module to the thermal pad.

16. The method of claim 15, wherein:
the thermal pad further comprises a fluid containing layer for containing the TTM fluid, the fluid containing layer configured for circulating the TTM fluid within the fluid containing layer, and
one or more fluid containing layer slits are arranged in the fluid containing layer, the fluid containing layer slits coinciding with the one or more slits of the insulating layer.

17. The method of claim 15, further comprising causing the patient contact surface to conform to a contour of a body part of the patient, wherein:
the body part is in contact with the patient contact surface, and
conforming of the patient contact surface to the contour is facilitated by the one or more slits.

18. The method of claim 15, wherein:
the thermal pad further comprises a flexible fabric cover, and
the flexible fabric cover further facilitates the stretching of the pad.

19. The method of claim 18, wherein the flexible fabric cover has a soft texture configured to reduce pressure on skin of the patient from an edge or surface of the pad.

20. The method of claim 18, wherein the flexible fabric cover comprises one or more of nylon, polychloroprene, elastane, latex, isoprene, polyisoprene, elastolefin, polybutadiene, nitrile rubber, butyl rubber, or a loose-woven fabric.

21. The method of claim 18, wherein the flexible fabric cover comprises
an exterior border portion surrounding a perimeter of the pad, and
a slit-covering portion coinciding with the one or more slits in the insulating layer.

22. The method of claim 18, further comprising causing the patient contact surface to conform to a contour of a body part of the patient, wherein:
the body part is in contact with the patient contact surface, and
conforming of the patient contact surface to the contour is facilitated by the flexible fabric cover.

23. The method of claim 18, wherein:
the flexible fabric cover coincides with the one or more slits in the insulating layer, and
an edge guard surrounds a perimeter of the pad, the edge guard configured to reduce pressure from an edge or surface of the pad on skin of the patient.

24. The method of claim 23, wherein the edge guard comprises one or more gaps coinciding with the one or more slits of the insulating layer.

25. The method of claim 23, wherein the edge guard has one or more edge guard slits coinciding with the one or more slits of the insulating layer.

26. The method of claim 23, wherein the edge guard includes
a pliant edge guard cover, and
a cushion filling contained within the pliant edge guard cover.

27. The method of claim 23, wherein the edge guard comprises silicone or another pliant shock-absorbent material.

28. The method of claim 15, wherein:
the fluid containing layer includes a plurality of tortuous fluid flow paths, and
the one or more slits are arranged in portions of the insulating layer not coinciding with the fluid flow paths of the fluid containing layer.
